(19) United States
(12) Patent Application Publication
(10) Pub. No.: US 2025/0281197 A1
(43) Pub. Date: Sep. 11, 2025

(54) ULTRASONIC TREATMENT INSTRUMENT

(71) Applicant: OLYMPUS MEDICAL SYSTEMS CORP., Tokyo (JP)

(72) Inventors: Tsubasa NIIYAMA, Tokyo (JP); Marina ASAHINA, Sagamihara-shi (JP); Keisuke NAGAO, Tokyo (JP); Kaito SATO, Tokyo (JP)

(21) Appl. No.: 18/956,945

(22) Filed: Nov. 22, 2024

Related U.S. Application Data
(63) Continuation of application No. PCT/JP2024/009171, filed on Mar. 8, 2024.

(51) Int. Cl. A61B 17/32; A61B 17/00

(52) U.S. Cl. CPC A61B 17/320092; A61B 2017/00853; A61B 2017/320082; A61B 2017/320094

(57) ABSTRACT

An ultrasonic treatment instrument includes: an ultrasonic blade configured to supply ultrasonic vibration and a high frequency current to a living tissue, respectively; a jaw configured to open and close with respect to the ultrasonic blade; a holder provided in the jaw to be swingable with respect to the jaw; and an abutment portion provided in the holder and made of a first resin material, the abutment portion being configured to abut against the ultrasonic blade when the jaw is closed with respect to the ultrasonic blade, at least a part of the holder being made of a second resin material, and the holder including an electrode configured to supply the high frequency current on at least a part of a contact surface that is included in the holder, the contact surface being configured to be in contact with the living tissue.

### CROSS REFERENCES TO RELATED APPLICATIONS

This application is a continuation of International Application No. PCT/JP2024/009171, filed on Mar. 8, 2024, the entire contents of which are incorporated herein by reference.

### BACKGROUND

### 1. Technical Field

The present disclosure relates to an ultrasonic treatment instrument.

### 2. Related Art

In the related art, there has been known an ultrasonic treatment instrument that supplies ultrasonic vibration and a high frequency current as treatment energy to a site (hereinafter, referred to as a treatment target) to be treated in a living tissue to treat the treatment target (see, for example, US 2021/0196334 A).

The ultrasonic treatment instrument described in US 2021/0196334 A includes an ultrasonic blade, a jaw, and an abutment portion described below.

The ultrasonic blade supplies the ultrasonic vibration and the high frequency current to the treatment target, respectively.

The jaw opens and closes with respect to the ultrasonic blade. In addition, the jaw is provided with an electrode that supplies the high frequency current to the treatment target.

The abutment portion is made of a resin material and is provided on the jaw. The abutment portion abuts against the ultrasonic blade when the jaw is closed with respect to the ultrasonic blade.

### SUMMARY

In some embodiments, an ultrasonic treatment instrument includes: an ultrasonic blade configured to supply ultrasonic vibration and a high frequency current to a living tissue, respectively; a jaw configured to open and close with respect to the ultrasonic blade; a holder provided in the jaw to be swingable with respect to the jaw; and an abutment portion provided in the holder and made of a first resin material, the abutment portion being configured to abut against the ultrasonic blade when the jaw is closed with respect to the ultrasonic blade, at least a part of the holder being made of a second resin material, and the holder including an electrode configured to supply the high frequency current on at least a part of a contact surface that is included in the holder, the contact surface being configured to be in contact with the living tissue.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram for describing a configuration of a distal end portion in an ultrasonic treatment instrument;

FIG. 3 is a diagram for describing a configuration of the distal end portion in the ultrasonic treatment instrument;

FIG. 4 is a diagram for describing a configuration of a jaw;

FIG. 5 is a diagram for describing a configuration of a holder; and

FIG. 6 is a diagram for describing a modification of the embodiment.

### DETAILED DESCRIPTION

Hereinafter, modes for carrying out the present invention (hereinafter, embodiments) will be described with reference to the drawings. The present invention is not limited by the embodiments described below. Furthermore, in the description of the drawings, the same portions are denoted by the same reference numerals.

### Schematic Configuration of Treatment System

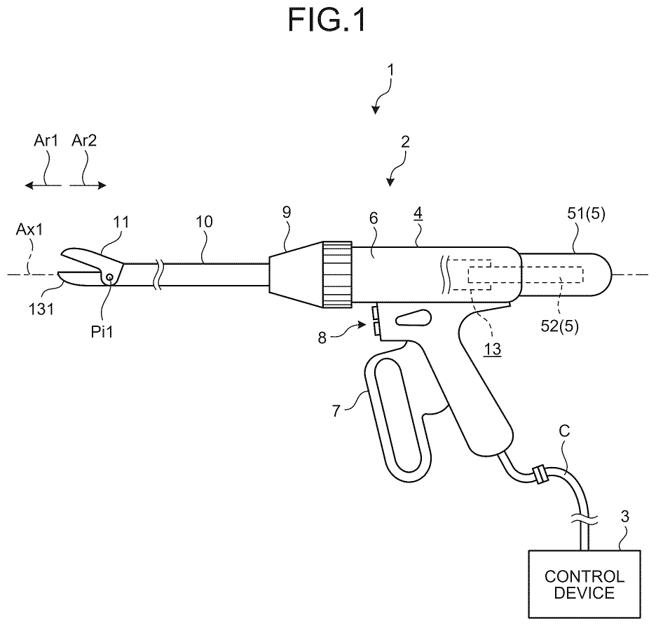

FIG. 1 is a diagram illustrating a treatment system 1 according to an embodiment.

The treatment system 1 applies treatment energy to a site to be treated (hereinafter, referred to as a treatment target) in a living tissue to treat the treatment target. The treatment energy in the present embodiment is ultrasonic energy and high frequency energy. In addition, the treatment that can be performed by the treatment system 1 according to the present embodiment is treatment such as coagulation (sealing) of the treatment target or incision of the treatment target. The coagulation and incision may be performed simultaneously. As illustrated in FIG. 1, the treatment system 1 includes a treatment instrument 2 and a control device 3.

### Configuration of Treatment Instrument

Hereinafter, one side along a central axis Ax1 (FIG. 1) of an outer pipe 10 is referred to as a distal end side Ar1, and the other side is referred to as a proximal end side Ar2. In addition, a “width direction” described below is a direction orthogonal to the central axis Ax1 and an opening/closing direction of a jaw 11 with respect to a treatment unit 131, and means a direction orthogonal to a paper surface of FIG. 1.

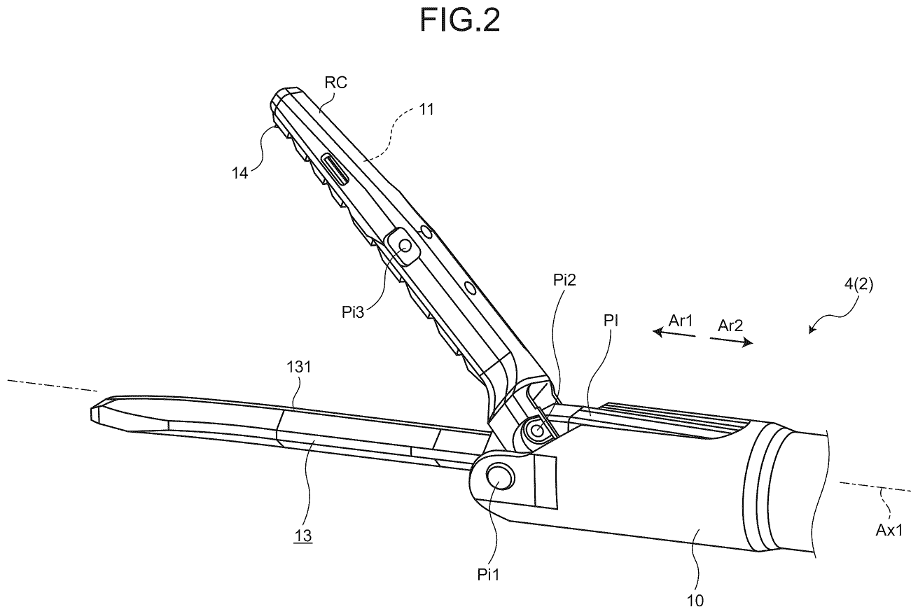

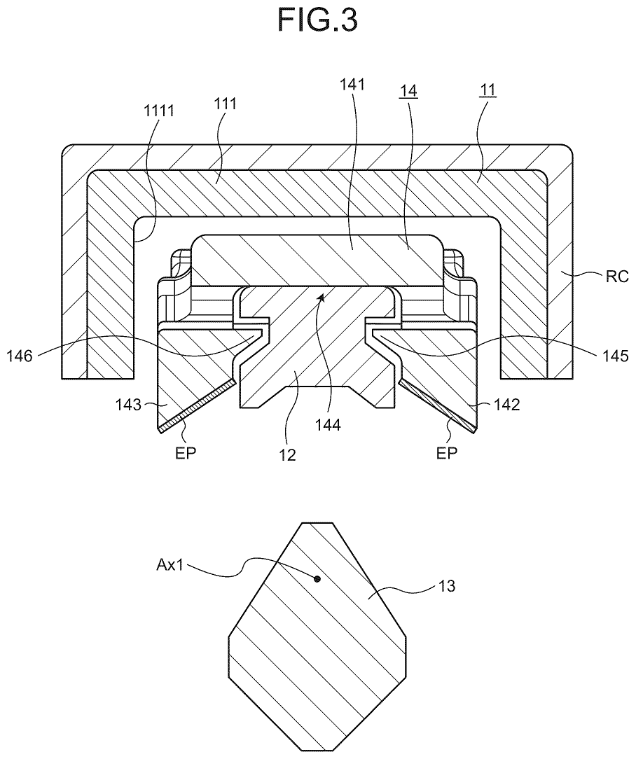

FIGS. 2 and 3 are diagrams for describing a configuration of a distal end portion in the treatment instrument 2. Specifically, FIG. 2 is a perspective diagram illustrating the distal end portion of the treatment instrument 2. FIG. 3 is a cross-sectional diagram of the distal end portion of the treatment instrument 2 taken along a plane orthogonal to the central axis Ax1.

The treatment instrument 2 is an ultrasonic treatment instrument. Then, the treatment instrument 2 treats a treatment target by applying ultrasonic energy and high frequency energy to the treatment target. As illustrated in FIG. 1, the ultrasonic treatment instrument 2 includes a handpiece 4 and an ultrasonic transducer portion 5.

As illustrated in FIGS. 1 to 3, the handpiece 4 includes a fixed handle 6 (FIG. 1), an operation handle 7 (FIG. 1), a switch 8 (FIG. 1), a rotary knob 9 (FIG. 1), the outer pipe 10 (FIGS. 1 and 2), the jaw 11, a holder 14 (FIGS. 2 and 3), an abutment portion 12 (FIG. 3), and the ultrasonic blade 13.

The fixed handle 6 is a portion that supports the entire treatment instrument 2 and is gripped by an operator (user) such as a surgeon.

The operation handle 7 is movably attached to the fixed handle 6 and receives an opening/closing operation by the operator such as a surgeon.

The switch 8 is provided in a state of being exposed to an outside of the fixed handle 6, and receives a treatment operation by the operator such as a surgeon.

The rotary knob 9 has a substantially cylindrical shape coaxial with the central axis Ax1, and is provided on the distal end side Ar1 of the fixed handle 6. Then, the rotary knob 9 receives a rotation operation by the operator such as a surgeon. By the rotation operation, the rotary knob 9 rotates about the central axis Ax1 with respect to the fixed handle 6. The outer pipe 10, the jaw 11, the holder 14, the abutment portion 12, and the ultrasonic blade 13 rotate about the central axis Ax1 by the rotation of the rotary knob 9.

The outer pipe 10 has a tubular shape and corresponds to a pipe. In the present embodiment, the outer pipe 10 is a cylindrical pipe made of an electrically conductive material such as metal.

In the outer pipe 10, a first pin Pi1 (FIGS. 1 and 2) having a cylindrical shape extending in a direction orthogonal to the paper surface of FIG. 1 and engaging with the jaw 11 and pivotally supporting the jaw 11 in a rotatable manner is fixed to an end portion on the distal end side Ar1. In the present embodiment, the first pin Pi1 is made of an electrically conductive material such as metal.

An outer peripheral surface of the outer pipe 10 is covered with an electrically insulating outer tube (not illustrated). Further, a tubular inner pipe PI (FIG. 2) that moves forward and backward along a longitudinal direction of the outer pipe 10 according to the opening/closing operation to the operation handle 7 by the operator such as a surgeon is inserted into the outer pipe 10. A second pin Pi2 (FIG. 2) having a cylindrical shape extending in a direction orthogonal to the paper surface of FIG. 1 and engaging with the jaw 11 is fixed to an end portion of the inner pipe PI on the distal end side Ar1. In the present embodiment, the second pin Pi2 is disposed on an upper side (a side on which a jaw main body 111 is disposed with respect to the treatment unit 131) in FIG. 2 with respect to the first pin Pi1.

The jaw 11 is coupled to the outer pipe 10 by the first pin Pi1. The jaw 11 is coupled to the inner pipe PI by the second pin Pi2. Then, the jaw 11 rotates about the first pin Pi1 with respect to the outer pipe 10 in conjunction with the forward and backward movement of the inner pipe PI according to the opening/closing operation to the operation handle 7 by the operator such as a surgeon. As a result, the jaw 11 is opened and closed with respect to the treatment unit 131 which is the end portion of the ultrasonic blade 13 on the distal end side, and can grip a treatment target with the treatment unit 131 therebetween.

Note that the treatment instrument 2 may be a push-close type or a pull-close type.

The push-close type has the following configuration.

The jaw 11 rotates about the first pin Pi1 in a direction approaching the treatment unit 131 in conjunction with the movement of the inner pipe PI toward the distal end side Ar1. That is, the jaw 11 is closed with respect to the treatment unit 131. The jaw 11 rotates about the first pin Pi1 in a direction away from the treatment unit 131 in conjunction with the movement of the inner pipe PI toward the proximal end side Ar2. That is, the jaw 11 opens with respect to the treatment unit 131.

The pull-close type has the following configuration.

The jaw 11 rotates about the first pin Pi1 in the direction approaching the treatment unit 131 in conjunction with the movement of the inner pipe PI toward the proximal end side Ar2. That is, the jaw 11 is closed with respect to the treatment unit 131. The jaw 11 rotates about the first pin Pi1 in a direction away from the treatment unit 131 in conjunction with the movement of the inner pipe PI toward the distal end side Ar1. That is, the jaw 11 opens with respect to the treatment unit 131.

A detailed configuration of the jaw 11 will be described in “Configuration of Jaw” described later.

The holder 14 has a cylindrical shape extending in a direction orthogonal to the paper surface of FIG. 1, and is pivotally supported with respect to the jaw 11 so as to be swingable about the central axis of a third pin Pi3 by the third pin Pi3 (FIG. 2) fixed to the jaw 11. That is, by making the holder 14 swingable about the central axis of the third pin Pi3, a position where a strongest force is applied to a treatment target when the treatment target is gripped between the jaw 11 and the treatment unit 131 is positioned at substantially the center of the jaw 11 in the longitudinal direction instead of the proximal end side Ar2 of the jaw 11. That is, a force is substantially uniformly applied to the treatment target gripped between the jaw 11 and the treatment unit 131. In the present embodiment, the third pin Pi3 is made of an electrically conductive material such as metal.

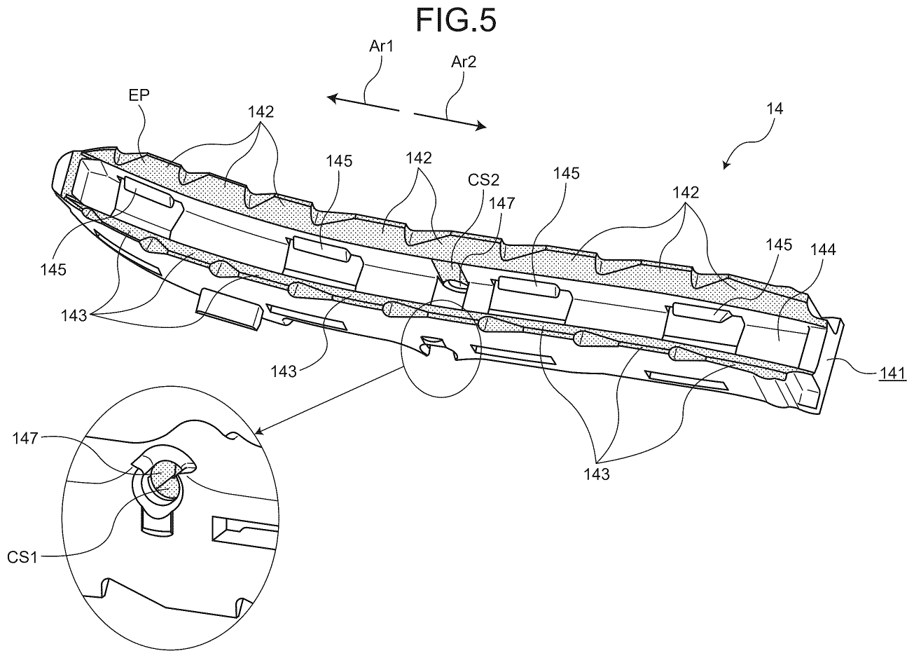

The holder 14 is provided with an electrode EP and first and second electrically conductive surfaces CS1 and CS2 (see FIG. 5).

A detailed configuration of the holder 14 will be described in “Configuration of Holder” described later. The configurations of the electrode EP and the first and second electrically conductive surfaces CS1 and CS2 will be described in “Configurations of Electrode and First and Second Electrically conductive Surfaces” described later.

The abutment portion 12 is made of a first resin material having electrical insulation properties and biocompatibility, for example, polytetrafluoroethylene (PTFE), and has a substantially rectangular parallelepiped shape extending along the longitudinal direction of the jaw 11 and the holder 14. Then, as illustrated in FIGS. 2 and 3, the abutment portion 12 is fixed to a surface of a holder main body 141 on a side of the treatment unit 131, and abuts against the treatment unit 131 when the jaw 11 is closed with respect to the treatment unit 131. The abutment portion 12 has a function of preventing the treatment unit 131 which is ultrasonically vibrated from being damaged by colliding with the jaw 11 when the incision of the treatment target by ultrasonic vibration is completed.

The ultrasonic blade 13 is made of an electrically conductive material and has an elongated shape extending along the central axis Ax1. In addition, the ultrasonic blade 13 is inserted into the inner pipe PI in a state where the treatment unit 131 protrudes to the outside. At this time, an end portion of the ultrasonic blade 13 on the proximal end side Ar2 is mechanically coupled to an ultrasonic transducer 52 configuring the ultrasonic transducer portion 5 as illustrated in FIG. 1. Then, the ultrasonic blade 13 transmits ultrasonic vibration generated by the ultrasonic transducer portion 5 from the end portion on the proximal end side Ar2 to the treatment unit 131. The ultrasonic vibration is longitudinal vibration that vibrates in a direction along the central axis Ax1. In the ultrasonic blade 13, an outer peripheral surface excluding the treatment unit 131 is covered with an electrically insulating inner tube.

As illustrated in FIG. 1, the ultrasonic transducer portion 5 includes a transducer (TD) case 51 and the ultrasonic transducer 52.

The TD case 51 supports the ultrasonic transducer 52 and is detachably coupled to the fixed handle 6.

The ultrasonic transducer 52 generates ultrasonic vibration under the control of the control device 3. In the present embodiment, the ultrasonic transducer 52 is configured by a bolted Langevin transducer (BLT).

### Configuration of Control Device

The control device 3 integrally controls an operation of the treatment instrument 2 via an electric cable C (FIG. 1).

Specifically, the control device 3 detects a treatment operation on the switch 8 by the operator such as a surgeon via the electric cable C. Then, when detecting the treatment operation, the control device 3 applies treatment energy to the treatment target gripped between the jaw 11 and the treatment unit 131 via the electric cable C. That is, the control device 3 treats the treatment target.

For example, when applying ultrasonic energy to the treatment target, the control device 3 supplies drive power to the ultrasonic transducer 52 via the electric cable C. As a result, the ultrasonic transducer 52 generates longitudinal vibration (ultrasonic vibration) that vibrates in a direction along the central axis Ax1. In addition, the treatment unit 131 vibrates with a desired amplitude by the longitudinal vibration. Then, the ultrasonic vibration is supplied from the treatment unit 131 to the treatment target gripped between the jaw 11 and the treatment unit 131. The ultrasonic energy is applied from the treatment unit 131 to the treatment target.

Furthermore, for example, when applying high frequency energy to the treatment target, the control device 3 supplies high frequency power between the electrode EP provided on the holder 14 and the ultrasonic blade 13 via the electric cable C or the like. When the high frequency power is supplied between the electrode EP and the ultrasonic blade 13, a high frequency current is supplied to the treatment target gripped between the jaw 11 and the treatment unit 131. In other words, the high frequency energy is applied to the treatment target.

### Configuration of Jaw

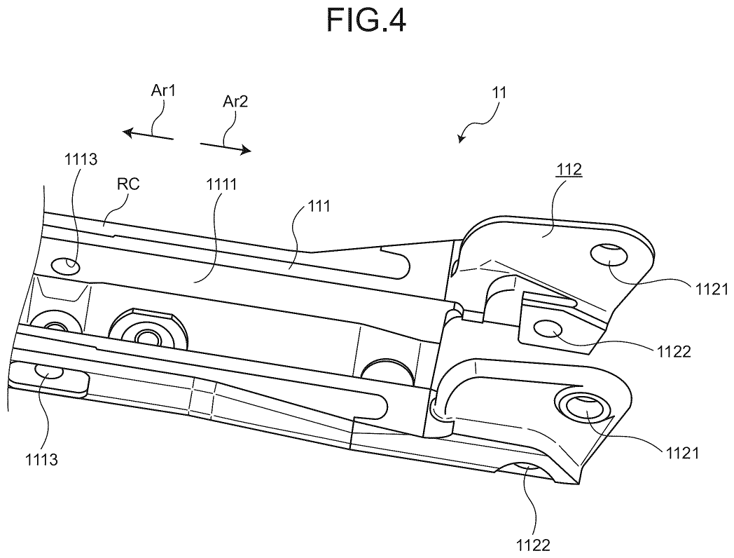

FIG. 4 is a diagram for describing a configuration of the jaw 11. Specifically, FIG. 4 is a perspective diagram of the jaw 11 as viewed from the side of the treatment unit 131. In FIG. 4, illustration of the distal end portion is omitted for convenience of description.

The jaw 11 is made of an electrically conductive material. As illustrated in FIG. 4, the jaw 11 is a member in which a jaw main body 111 and a pair of bearing portions 112 are integrally formed.

The jaw main body 111 is formed of a substantially elongated plate body.

In the jaw main body 111, as illustrated in FIG. 4, a recess 1111 extending from the proximal end toward the distal end side Ar1 along the longitudinal direction of the jaw main body 111 is provided on a surface on the side of the treatment unit 131.

In side wall portions on both sides in the width direction of the jaw main body 111 configuring the recess 1111, as illustrated in FIG. 4, through-holes 1113 penetrating the front and back of the side wall portions are provided at positions substantially at the center of the jaw 11 in the longitudinal direction. The third pin Pi3 inserted into the through-holes 1113 is fixed by welding.

In addition, a cover RC (FIGS. 2 to 4) made of an electrically insulating resin is integrally formed on a back side surface of the jaw main body 111 separated from the treatment unit 131 in a state of covering the back side surface. In the present embodiment, the cover RC is insert-molded with respect to the jaw main body 111, but the disclosure is not limited thereto. For example, a configuration in which the cover RC is fixed to the jaw main body 111 by snap-fitting or a metal pin may be adopted.

The pair of bearing portions 112 are provided at end portions of the jaw main body 111 on the proximal end side Ar2, and are configured by plate bodies facing each other in the width direction of the jaw main body 111. The pair of bearing portions 112 have the same configuration. Therefore, only the configuration of one bearing portion 112 will be described below.

The bearing portion 112 is provided with first and second insertion holes 1121 and 1122 penetrating the front and back, respectively. The bearing portion 112 is coupled to the outer pipe 10 by inserting the first pin Pi1 into the first insertion hole 1121. The bearing portion 112 is coupled to the inner pipe PI by inserting the second pin Pi2 into the second insertion hole 1122.

### Configuration of Holder 14

FIG. 5 is a diagram for describing a configuration of the holder 14. Specifically, FIG. 5 is a perspective diagram of the holder 14 as viewed from the side of the treatment unit 131. In FIG. 5, dots are added to the electrode EP and the first and second electrically conductive surfaces CS1 and CS2 for convenience of description.

The holder 14 is made of a second resin material having electrical insulation properties and biocompatibility, for example, polyether ether ketone (PEEK) or polyphenyl sulfone (PPSU). As illustrated in FIG. 5, the holder 14 is a member in which a holder main body 141, a plurality of first tooth portions 142, and a plurality of second tooth portions 143 are integrally formed.

The holder main body 141 is formed of an elongated plate body. In addition, an outer shape of the holder main body 141 is set to be substantially the same as an inner surface shape of the recess 1111.

As illustrated in FIG. 5, the plurality of first tooth portions 142 respectively protrude from one side in the width direction of the surface of the holder main body 141 on the side of the treatment unit 131 toward the side of the treatment unit 131, and are arranged side by side along the longitudinal direction of the holder main body 141.

As illustrated in FIG. 5, the plurality of second tooth portions 143 respectively protrude from the other side in the width direction of the surface of the holder main body 141 on the side of the treatment unit 131 toward the side of the treatment unit 131, and are arranged side by side along the longitudinal direction of the holder main body 141.

Here, on the surface of the holder main body 141 on the side of the treatment unit 131, at the center portion in the width direction located between the plurality of first tooth portions 142 and the plurality of second tooth portions 143, as illustrated in FIG. 5, a recess 144 that is recessed on a side separated from the treatment unit 131 and extending along the longitudinal direction of the holder main body 141 is provided. Further, claw portions 145 and 146 (FIGS. 3 and 5) protruding toward the center in the width direction and extending along the longitudinal direction of the holder main body 141 are respectively provided in side wall portions on both sides in the width direction of the holder main body 141 configuring the recess 144. Then, the abutment portion 12 is mechanically fixed to the holder 14 by being locked to the claw portions 145 and 146. That is, the abutment portion 12 is provided at the center portion of the holder 14 in the width direction. Further, in the side wall portions on both sides in the width direction of the holder main body 141 configuring the recess 144, insertion holes 147 which respectively penetrate through the front and back of the side wall portions and through which the third pin Pi3 is inserted are provided.

### Configurations of Electrode and First and Second Electrically Conductive Surfaces

As illustrated in FIG. 5, the electrode EP is provided on each of inner surfaces of the plurality of first tooth portions 142 in the width direction (on the side of the second tooth portion 143), distal end surfaces of the first tooth portions 142, inner surfaces of the plurality of second tooth portions 143 in the width direction (the side of the first tooth portion 142), and distal end surfaces of the second tooth portions 143, respectively. In addition, a coating material having non-adhesiveness to the treatment target is applied onto the electrode EP. The coating material is an extremely thin coating material including fluorine or silicon and having a thickness of about several hundred nm to several μm.

As illustrated in FIG. 5, the first electrically conductive surface CS1 is provided on an inner surface of the insertion hole 147.

As illustrated in FIG. 5, the second electrically conductive surface CS2 is provided on the side wall portion of the recess 144, and electrically couples the electrode EP and the first electrically conductive surface CS1.

The electrode EP and the first and second electrically conductive surfaces CS1 and CS2 described above are formed by the following three-dimensional plating processing.

Specifically, the holder 14 made of the second resin material is irradiated with a laser at a predetermined position. Thereafter, the electrode EP and the first and second electrically conductive surfaces CS1 and CS2 are formed at the laser irradiation position by electroless plating. A thickness of the electrode EP and the first and second electrically conductive surfaces CS1 and CS2 is, for example, about several μm. In addition, a formed product produced by such three-dimensional plating processing is called a molded interconnect device (MID) as a product in which an electrode circuit is formed on the surface of a three-dimensional resin molded article. The electrode EP and the first and second electrically conductive surfaces CS1 and CS2 are not limited to those formed by the above-described three-dimensional plating processing, and those formed by other methods may be adopted.

Then, when high frequency power is supplied to the electrode EP in order to apply high frequency energy to the treatment target, the high frequency power is supplied following an electric path of the electric cable C to the outer pipe 10 to the first pin Pi1 to the jaw 11 to the third pin Pi3 to the first electrically conductive surface CS1 to the second electrically conductive surface CS2 and to the electrode EP.

According to the present embodiment described above, the following effects are obtained.

In the treatment instrument 2 according to the present embodiment, at least a part of the holder 14 is made of the second resin material. The holder 14 is provided with the electrode EP described above. Therefore, rigidity of the holder 14 can be made smaller than that in a case where it is made of a metal material. That is, even when the abutment portion 12 is worn by repeatedly outputting the ultrasonic vibration, a distance between the ultrasonic blade 13 and the holder 14 decreases, and the ultrasonic blade 13 and the holder 14 come into contact with each other, an influence on the ultrasonic blade 13 can be reduced.

Therefore, the treatment instrument 2 according to the present embodiment can reduce an influence on a life of the ultrasonic blade 13.

In particular, the electrode EP is formed by three-dimensional plating processing. Therefore, even when the holder 14 is made of the second resin material, the electrode EP can be easily formed at a specific position in the holder 14.

In addition, a coating material having non-adhesiveness to the treatment target is provided on the electrode EP. Therefore, it is possible to prevent the treatment target from adhering to the electrode EP and to favorably treat the treatment target.

### Other Embodiments

Although the embodiment for carrying out the present invention have been described so far, the present invention should not be limited only by the above-described embodiment.

In the above-described embodiment, the outer pipe 10 is adopted as the electric path from the electric cable C to the electrode EP, but the present invention is not limited thereto, and the inner pipe PI may be adopted instead of the outer pipe 10.

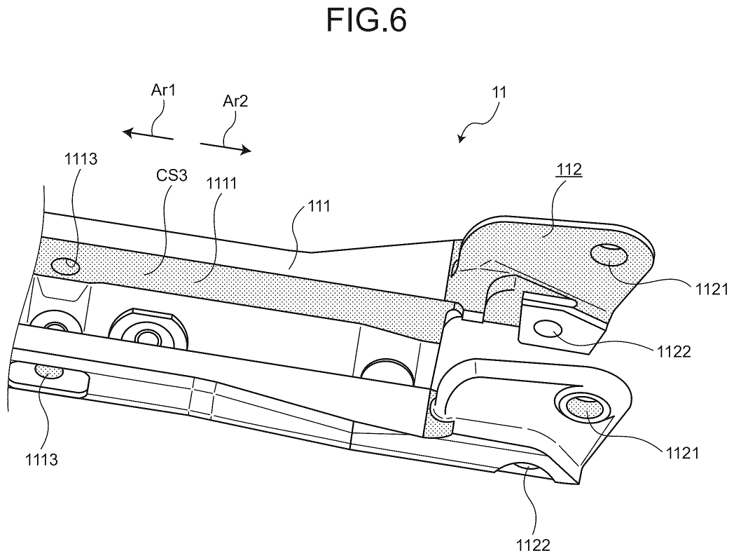

FIG. 6 is a diagram for describing a modification of the embodiment. Specifically, FIG. 6 is a diagram corresponding to FIG. 4. In FIG. 6, dots are added to a third electrically conductive surface CS3 for convenience of description.

In the above-described embodiment, the jaw 11 is made of an electrically conductive material, but the present invention is not limited thereto, and may be made of a third resin material. As the third resin material, a resin material similar to the second resin material described in the above-described embodiment can be adopted. In this case, as illustrated in FIG. 6, the jaw 11 is provided with the third electrically conductive surface CS3 in order to secure the electric path from the first pin Pi1 to the third pin Pi3.

The third electrically conductive surface CS3 is provided on the inner surface of the first insertion hole 1121, the inner surface of the through-hole 1113, the surface of the bearing portion 112 facing the other bearing portion 112, and the side wall portion of the recess 1111, and secures an electric path from the first pin Pi1 inserted into the first insertion hole 1121 to the third pin Pi3 inserted into the through-hole 1113.

The third electrically conductive surface CS3 described above is formed by three-dimensional plating processing similarly to the electrode EP and the first and second electrically conductive surfaces CS1 and CS2 described in the above-described embodiment. The third electrically conductive surface CS3 is not limited to one formed by the above-described three-dimensional plating processing, and one formed by another method may be adopted.

Even in a case where the configuration of the present modification illustrated in FIG. 6 is adopted, the same effects as those of the above-described embodiment are obtained.

According to an ultrasonic treatment instrument of the disclosure, an influence on a life of an ultrasonic blade can be reduced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment instrument comprising:
an ultrasonic blade configured to supply ultrasonic vibration and a high frequency current to a living tissue, respectively;
a jaw configured to open and close with respect to the ultrasonic blade;
a holder provided in the jaw to be swingable with respect to the jaw; and
an abutment portion provided in the holder and made of a first resin material, the abutment portion being configured to abut against the ultrasonic blade when the jaw is closed with respect to the ultrasonic blade,
at least a part of the holder being made of a second resin material, and
the holder including an electrode configured to supply the high frequency current on at least a part of a contact surface that is included in the holder, the contact surface being configured to be in contact with the living tissue.

2. The ultrasonic treatment instrument according to claim 1, wherein
the abutment portion is provided at a center portion of the holder in a width direction, and
the electrode is provided on at least a part of each of contact surfaces located on both sides of the abutment portion in the width direction.

3. The ultrasonic treatment instrument according to claim 1, wherein the second resin material is polyether ether ketone or polyphenyl sulfone.

4. The ultrasonic treatment instrument according to claim 1, wherein the electrode is formed by three-dimensional plating processing.

5. The ultrasonic treatment instrument according to claim 1, further comprising:
a cylindrical pin attached to the jaw and swingably supporting the holder, wherein
the electrode is electrically coupled to the jaw via the pin.

6. The ultrasonic treatment instrument according to claim 5, wherein
the holder is provided with an insertion hole through which the pin is inserted, and
an inner surface of the insertion hole is provided with a first electrically conductive surface configured to electrically couple the pin and the electrode.

7. The ultrasonic treatment instrument according to claim 6, wherein
the holder is provided with a second electrically conductive surface configured to electrically couple the first electrically conductive surface and the electrode.

8. The ultrasonic treatment instrument according to claim 1, wherein a coating material having non-adhesiveness to the living tissue is provided on the electrode.

9. The ultrasonic treatment instrument according to claim 6, wherein the jaw is made of a third resin material and includes a third electrically conductive surface configured to be electrically coupled to the pin.

10. The ultrasonic treatment instrument according to claim 6, wherein
the first resin material is polytetrafluoroethylene (PTFE),
the second resin material is polyether ether ketone (PEEK) or polyphenyl sulfone (PPSU), and
the third resin material is polyether ether ketone (PEEK) or polyphenyl sulfone (PPSU).
